(19) United States
(12) Patent Application Publication
(10) Pub. No.: US 2025/0185986 A1
(43) Pub. Date: Jun. 12, 2025

(54) METHODS FOR PROVIDING MORE EFFECTIVE COMPRESSION THERAPY

(71) Applicant: Wound Pros Technology, Inc., Los Angeles, CA (US)

(72) Inventor: Daniel Lee Hallman, Los Angeles, CA (US)

(73) Assignee: Wound Pros Technology, Inc., Los Angeles, CA (US)

(21) Appl. No.: 18/974,202

(22) Filed: Dec. 9, 2024

Related U.S. Application Data
(60) Provisional application No. 63/607,636, filed on Dec. 8, 2023.

(51) Int. Cl. A61B 5/00; A61B 5/0295

(52) U.S. Cl. CPC A61B 5/4848; A61B 5/0295; A61B 5/7267

(57) ABSTRACT

This disclosure provides a method for providing more effective compression therapy, for example, by including pre and post volume plethysmography as indicators in addition to Ankle-Brachial Index (ABI)/Toe-Brachial Index (TBI) and pulse volume recording (PVR) to evaluate safety and effectiveness of compression therapy.

### CROSS-REFERENCE TO RELATED APPLICATIONS

This application claims priority under 35 U.S.C. § 119(e) to U.S. Provisional Patent Application No. 63/607,636, filed Dec. 8, 2024. The foregoing application is incorporated by reference herein in its entirety.

### FIELD OF THE INVENTION

The present invention relates generally to methods for providing more effective compression therapy.

### BACKGROUND OF THE INVENTION

Venous ulcers are due to abnormal vein function. People may inherit a tendency for abnormal veins. Common causes of damaged veins include blood clots, injury, aging, and obesity. Symptoms include swelling, achiness, and tiredness in the legs. Usually a red, irritated skin rash develops into an open wound. Treatment includes leg elevation, compression, and wound care. Sometimes surgery is needed.

About 70% of all leg ulcers are venous ulcers. Of the approximately 7 million people in the United States with venous insufficiency, approximately 1 million develop venous leg ulcers. The cost of venous leg ulcers is estimated to be $1 billion per year in the United States, and the average cost per patient exceeds $40,000. Venous leg ulcer occurs secondary to underlying venous disease whereby blockage or valve damage leading to valvar insufficiency of the superficial, deep or perforating veins leads to venous hypertension. The ulcer usually presents within the region of the leg just above the ankle. In general, venous ulcers are treated with compression stockings, wraps or bandages. Graduated compression can reduce the elevated pressures in the superficial veins. Compression may also improve the competence of the valves.

Venous leg ulcers (VLUs) pose a significant challenge in patients with peripheral arterial disease (PAD), often requiring individualized treatment plans. The standard of care involves compression therapy, but its safety and efficacy depend on accurate vascular assessment. Thus, there is a pressing need for an improved therapy for venous leg ulcers.

### SUMMARY OF THE INVENTION

In one aspect, this disclosure provides a method for providing more effective compression therapy, for example, by including pre and post volume plethysmography as indicators in addition to Ankle-Brachial index (ABI)/Toe-Brachial Index (TBI). and pulse volume recording (PVR) to evaluate safety and effectiveness of compression therapy.

In one aspect, this disclosure provides a method of providing a compression therapy to a patient who is suffering from venous leg ulcers. In some embodiments, the method comprises: (a) identifying a venous condition in a patient as having venous leg ulcers and a peripheral arterial disease; (b) determining an arterial condition of the patient in real-time at least by digital volume plethysmography before and after applying the compression therapy to measure volume changes in the body caused by blood flow using one or more sensors or blood pressure cuffs; and (c) applying compression pressure to a part of the body of the patient to improve blood circulation based on real-time data of both the venous condition and the arterial condition of the patient, wherein assessment of the arterial condition guides the compression therapy such that the compression therapy does not cause arterial compromise exceeding a pre-determined threshold level.

In some embodiments, the digital volume plethysmography provides real-time vascular assessment in the patient in response to the compression therapy. In some embodiments, the arterial compromise comprises a blood flow change under compression.

In some embodiments, the digital volume plethysmography comprises pre and post digital volume plethysmography. In some embodiments, the digital volume plethysmography is measured in form of digital pressures and/or waveforms.

In some embodiments, the method comprises modifying an amount of compression pressure based on disease progression of venous leg ulcers.

In some embodiments, the compression pressure is applied through a compression bandage or stocking. In some embodiments, the compression pressure is applied by a gradient compression system. In some embodiments, the amount of compression pressure applied to the patient is from 20 mmHg to 30 mmHg or from 30 mmHg to 40 mmHg.

In some embodiments, the method further comprises determining an amount of compression pressure to the body of the patient by a trained model. In some embodiments, the trained model comprises a machine learning model. In some embodiments, the machine learning model comprises a supervised or unsupervised machine learning model. In some embodiments, the machine learning model comprises Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression. Support Vector Regression, deep learning approach, Neural Networks. Convolutional Neural Network (CNNs), Recurrent Neural Networks (RNNs), Gated Recurrent Units (GRUs). Long Short Term Memory Networks (LSTMs), Generative Models. Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or a combination thereof.

In some embodiments, the part of the body of the patient comprises a foot of the patient.

In some embodiments, the method is performed in a point-of-care setting. In some embodiments, the method is performed in a mobile care setting.

In some embodiments, the step of determining the arterial condition comprises determining the arterial condition based on Ankle-Brachial Index (ABI)/Toe-Brachial Index (TBI) and/or by pulse volume recording. In some embodiments, determining the arterial condition is not performed based on Ankle-Brachial Index (ABI)/Toe-Brachial Index (TBI). In some embodiments, determining the arterial condition is not performed by pulse volume recording.

In another aspect, this disclosure provides a system for providing a compression therapy to a patient who is suffering from venous leg ulcers. In some embodiments, the system comprises one or more processors configured to implement the method as described herein.

In yet another aspect, this disclosure provides a non-transitory computer-readable medium comprising instructions that, when executed by one or more processors, cause the one or more processors to perform the method as described herein.

In yet another aspect, this disclosure additionally provides a non-transitory computer-readable medium comprising instructions that, when executed by one or more processors, cause the one or more processors to perform the method as described herein.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features is not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### DETAILED DESCRIPTION OF THE INVENTION

Venous leg ulcers are chronic wounds that result from prolonged venous insufficiency, primarily affecting older adults. These ulcers develop due to poor blood flow in the veins, leading to tissue breakdown, especially in the lower extremities. Over time, this can cause significant disability and complications, such as infection and delayed healing. As populations age globally, the incidence of venous leg ulcers continues to rise. The condition requires careful management, including proper diagnosis, wound care, and prevention strategies to avoid recurrence.

Venous leg ulcers (VLUs) pose a significant challenge in patients with peripheral arterial disease (PAD), often requiring individualized treatment plans. While it is well-established that compression therapy is the gold standard for venous leg ulcers, its safety and efficacy depend on accurate vascular assessment. It is also recognized that many patients with VLUs may have mixed disease, meaning they suffer from both venous and arterial conditions. In these cases, the challenge lies in ensuring that compression therapy does not exacerbate underlying arterial insufficiency. While Ankle-Brachial Index (ABI)/Toe-Brachial Index (TBI) and pulse volume recording (PVR) are standard methods for vascular assessment, they do not provide a real-time assessment of the effects of compression therapy on the wound and vascular dynamics, particularly in a mobile point-of-care setting.

One novel and innovative aspect of the approach as describe herein is the incorporation of pre- and post-digital volume plethysmography to assess the immediate impact of compression therapy in real time. This addition allows clinicians to quickly evaluate whether compression is causing harm, if the right amount of compression is being applied, and whether arterial perfusion is being compromised during the application of compression-critical information that cannot be obtained solely from traditional vascular assessments.

This approach fills a critical gap by providing an immediate, data-driven response in the point-of-care setting, particularly in mobile wound care environments, where access to diagnostic resources can be limited. The ability to monitor vascular changes in real time allows clinicians to adjust treatment protocols dynamically, ensuring that patients receive compression therapy that is both safe and effective. It is especially important for patients with mixed disease, where the wrong amount of compression can worsen arterial perfusion and potentially lead to more severe complications.

By integrating digital volume plethysmography into clinical practice, this approach offers an unprecedented level of precision in managing compression therapy, particularly for diabetic patients or those with PAD. It enables clinicians to make informed decisions quickly, which can greatly reduce the risk of worsening arterial perfusion while optimizing wound healing. This real-time, mobile assessment tool can revolutionize the way to manage VLUs, ensuring that compression therapy is delivered safely and effectively across diverse patient populations.

While compression therapy remains the standard for treating VLUs, the use of pre- and post-digital volume plethysmography is a novel advancement that adds a crucial layer of safety and efficacy. This approach could transform wound care management, particularly in mobile care settings, by helping to deliver personalized, data-driven treatment tailored to the specific needs of patients with mixed disease.

In one aspect, this disclosure provides a method of providing a compression therapy to a patient who is suffering from venous leg ulcers. In some embodiments, the method comprises: (a) identifying a venous condition in a patient as having venous leg ulcers and a peripheral arterial disease; (b) determining an arterial condition of the patient in real-time at least by digital volume plethysmography before and after applying the compression therapy to measure volume changes in the body caused by blood flow using one or more sensors or blood pressure cuffs; and (c) applying compression pressure to a part of the body of the patient to improve blood circulation based on real-time data of both the venous condition and the arterial condition of the patient, wherein assessment of the arterial condition guides the compression therapy such that the compression therapy does not cause arterial compromise exceeding a pre-determined threshold level.

The method as disclosure involves combining venous condition identification (via, e.g., venous leg ulcers) with arterial condition determination using, e.g., digital volume plethysmography to adjust compression therapy in real-time. The approach of using pre- and post-plethysmography and incorporating vascular conditions in decision-making aligns with the need for tailored and safe compression therapy. The real-time data regarding the venous condition and the arterial condition is helpful in guiding compression therapy. For example, it can help to avoid arterial compromise, such that an appropriate amount of compression is application and not to exceed outlined thresholds or ranges for acceptable blood flow changes under compression. Due to incorporation of real-time vascular assessments (via, for example, digital volume plethysmography) before and after applying compression therapy, the disclosed method provides clinicians with actionable insights to prevent complications such as ischemic events or worsening ulcers.

Thus, the arterial condition assessment via digital volume plethysmography helps determine the safety of applying compression and adjust pressure accordingly to avoid worsening arterial perfusion. Notably, the disclosed method can be implemented in various settings, including a mobile care setting, a point of care setting, as well as non-invasive or low risk settings.

As used herein, “compression therapy” refers to a medical technique that uses specially designed garments or devices to apply pressure to the body, usually the limbs, to improve blood circulation and relieve a range of health issues. For treating ulcers, particularly venous leg ulcers, the recommended compression pressure is typically between 30-40 mmHg at the ankle; this strong compression helps promote healing by improving blood flow and reducing venous pressure in the affected area, making it the mainstay treatment for such ulcers. Strong compression, usually exceeding 30 mmHg at the ankle, is generally required for effective ulcer healing. Most compression stockings or bandages are designed with graduated compression, meaning the pressure is highest at the ankle and gradually decreases up the leg. Depending on the patient's condition, including arterial health, the compression pressure may need to be adjusted to avoid complications, as described in the disclosed approach. Compression therapy may not be suitable for patients with severe arterial disease, as excessive pressure can further restrict blood flow to the affected area. For some patients, factors such as mild arterial disease, neuropathy or cardiac failure render strong compression unsafe or painful and mild or moderate compression may be required (e.g., using inelastic compression).

Plethysmography is a test that measures volume changes in the body using sensors or blood pressure cuffs. The sensors or cuffs are attached to a plethysmograph, a machine that displays each pulse wave. Plethysmography is particularly effective at detecting changes caused by blood flow. Digital volume plethysmography is a method for measuring the rate of blood flow into and out of a fingertip at the same time. It can be used to evaluate digital pressures and waveforms.

Arterial conditions are vascular diseases that affect the arteries, which are the blood vessels that carry oxygen-rich blood from the heart to the body. Arterial conditions may include aneurysm, aortic dissection, Buerger's disease, carotid artery disease, claudication, intestinal artery disease, peripheral artery disease, Raynaud phenomenon, stroke, varicose veins, vasculitis, and the like. Arterial disorders affect the arteries, while venous disorders involve the veins, which return blood to the heart for more oxygen.

Venous conditions are a range of conditions that affect veins and can cause a variety of symptoms. Venous conditions may include varicose veins, spider veins, superficial thrombophlebitis, deep vein thrombosis (DVT), chronic venous insufficiency, pulmonary embolism, edema, skin discoloration, ulcers, and the like. Treatments for venous disease may include medications, compression stockings, bandages, lifestyle changes, and procedures or surgeries.

In some embodiments, the digital volume plethysmography provides real-time vascular assessment in the patient in response to the compression therapy. In some embodiments, the arterial compromise comprises a blood flow change under compression.

In some embodiments, the digital volume plethysmography comprises pre and post digital volume plethysmography. In some embodiments, the digital volume plethysmography is measured in form of digital pressures and/or waveforms.

In some embodiments, the method comprises modifying an amount of compression pressure based on disease progression of venous leg ulcers.

In some embodiments, the compression pressure is applied through a compression bandage or stocking. In some embodiments, the compression pressure is applied by a gradient compression system.

In some embodiments, the amount of compression pressure applied to the patient is from 20 mmHg to 30 mmHg or from 30 mmHg to 40 mmHg.

In some embodiments, the method further comprises determining an amount of compression pressure to the body of the patient by a trained model. In some embodiments, the trained model comprises a machine learning model. In some embodiments, the machine learning model comprises a supervised or unsupervised machine learning model. In some embodiments, the machine learning model comprises Deep Learning algorithm, Logistic Regression. Naive Bayes, Support Vector Machine, Decision Tree, Random Forest, Gradient Boosting, Regularizing Gradient Boosting, K-Nearest Neighbors, a continuous regression approach, Ridge Regression. Kernel Ridge Regression, Support Vector Regression, deep learning approach, Neural Networks, Convolutional Neural Network (CNNs), Recurrent Neural Networks (RNNs), Gated Recurrent Units (GRUs), Long Short Term Memory Networks (LSTMs), Generative Models. Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or a combination thereof.

In some embodiments, the part of the body of the patient comprises a foot of the patient.

In some embodiments, the method is performed in a point-of-care setting. In some embodiments, the method is performed in a mobile care setting.

In some embodiments, the step of determining the arterial condition comprises determining the arterial condition based on Ankle-Brachial Index (ABI)/Toe-Brachial Index (TBI) and/or by pulse volume recording. In some embodiments, determining the arterial condition is not performed based on Ankle-Brachial Index (ABI)/Toe-Brachial Index (TBI). In some embodiments, determining the arterial condition is not performed by pulse volume recording.

In another aspect, this disclosure provides a system for providing a compression therapy to a patient who is suffering from venous leg ulcers. In some embodiments, the system comprises one or more processors configured to implement the method as described herein.

In yet another aspect, this disclosure provides a non-transitory computer-readable medium comprising instructions that, when executed by one or more processors, cause the one or more processors to perform the method as described herein.

In yet another aspect, this disclosure additionally provides a non-transitory computer-readable medium comprising instructions that, when executed by one or more processors, cause the one or more processors to perform the method as described herein.

In some embodiments, the model comprises a machine learning model. As used herein, a “machine learning model,” a “model,” or a “classifier” refers to a set of algorithmic routines and parameters that can predict an output(s) for a process input based on a set of input features, with or without being explicitly programmed. A structure of the software routines (e.g., number of subroutines and relation between them) and/or the values of the parameters can be determined in a training process, which can use actual results of the process that is being modeled. Such systems or models are understood to be necessarily rooted in computer technology, and, in fact, cannot be implemented or even exist in the absence of computing technology. While machine learning systems utilize various types of statistical analyses, machine learning systems are distinguished from statistical analyses by virtue of the ability to learn without explicit programming and being rooted in computer technology. A neural network or an artificial neural network is one set of algorithms used in machine learning for modeling the data using graphs of neurons. Any network structure may be used. Any number of layers, nodes within layers, types of nodes (activations), types of layers, interconnections, learnable parameters, and/or other network architectures may be used. Machine training uses the defined architecture, training data, and optimization to learn values of the learnable parameters of the architecture based on the samples and ground truth of training data.

A typical machine learning pipeline may include building a machine learning model from a sample dataset (referred to as a “training set”), evaluating the model against one or more additional sample datasets (referred to as a “validation set” and/or a “test set”) to decide whether to keep the model and to benchmark how good the model is, and using the model in “production” to make predictions or decisions against live input data captured by an application service. For training the model to be applied as a machine-learned model, training data is acquired and stored in a database or memory. The training data is acquired by and gation, mining, loading from a publicly or privately formed collection, transfer, and/or access. Ten, hundreds, or thousands of samples of training data are acquired. The samples are from scans of different patients and/or phantoms. Simulation may be used to form the training data. The training data includes the desired output (ground truth), such as segmentation, and the input, such as protocol data and imaging data. In some embodiments, the training set will be used to create a single classifier using any now or hereafter-known methods. In other embodiments, a plurality of training sets will be created to generate a plurality of corresponding classifiers. Each of the plurality of classifiers can be generated based on the same or different learning algorithm that utilizes the same or different features in the corresponding one of the pluralities of training sets.

Once trained, the machine-learned or trained classifier is stored for later application. The training determines the values of the learnable parameters of the network. The network architecture, values of non-learnable parameters, and values of the learnable parameters are stored as the machine-learned network. Once stored, the machine-learned network may be fixed. The same machine-learned network may be applied to different patients, different scanners, and/or with different imaging protocols for the scanning. The machine-learned network may be updated. As additional training data is acquired, such as through application of the network for patients and corrections by experts to that output, the additional training data may be used to re-train or update the training. The training is performed by optimizing parameters of the model based on outputs of the model matching or not matching corresponding labels of the first labels and optionally the second labels when the first plurality of first data structures and optionally the second plurality of second data structures are input to the model. In some embodiments, the output of the model may include a probability of being in each of a plurality of states. The state with the highest probability can be taken as the state.

In some embodiments, the machine learning model may further include a supervised learning model. Supervised learning models may include different approaches and algorithms including analytical learning, artificial neural network, backpropagation, boosting (meta-algorithm), Bayesian statistics, case-based reasoning, decision tree learning, inductive logic programming, Gaussian process regression, genetic programming, group method of data handling, kernel estimators, learning automata, learning classifier systems, minimum message length (decision trees, decision graphs, etc.), multilinear subspace learning, naive Bayes classifier, maximum entropy classifier, conditional random field, Nearest Neighbor Algorithm, probably approximately correct learning (PAC) learning, ripple down rules, a knowledge acquisition methodology, symbolic machine learning algorithms, subsymbolic machine learning algorithms, support vector machines, Minimum Complexity Machines (MCM), random forests, ensembles of classifiers, ordinal classification, data pre-processing, handling imbalanced datasets, statistical relational learning, or Proaftn, a multicriteria classification algorithm, linear regression, logistic regression, deep recurrent neural network (e.g., long short term memory, LSTM), Bayes classifier, hidden Markov model (HMM), linear discriminant analysis (LDA), k-means clustering, density-based spatial clustering of applications with noise (DBSCAN), random forest algorithm, support vector machine (SVM), or any model described herein.

In some embodiments, the classifier may include a supervised or unsupervised Machine Learning or Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Gated Recurrent Units (GRUs), Long Short Term Memory Networks (LSTMs), Generative Models, Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or any combination thereof.

In some embodiments, the model may include a convolutional neural network (CNN). The CNN may include a set of convolutional filters configured to filter the first plurality of data structures and, optionally, the second plurality of data structures. The filter may be any filter described herein. The number of filters for each layer may be from 10 to 20, 20 to 30, 30 to 40, 40 to 50, 50 to 60, 60 to 70, 70 to 80, 80 to 90, 90 to 100, 100 to 150, 150 to 200, or more. The kernel size for the filters can be 2, 3, 4, 5, 6, 7, 8, 9, 10, 11, 12, 13, 14, 15, from 15 to 20, from 20 to 30, from 30 to 40, or more. The CNN may include an input layer configured to receive the filtered first plurality of data structures and, optionally, the filtered second plurality of data structures. The CNN may also include a plurality of hidden layers, including a plurality of nodes. The first layer of the plurality of hidden layers is coupled to the input layer. The CNN may further include an output layer coupled to a last layer of the plurality of hidden layers and configured to output an output data structure. The output data structure may include the properties.

In yet another aspect, this disclosure additionally provides a non-transitory computer-readable medium comprising instructions that, when executed by one or more processors, cause the one or more processors to perform the method as described herein.

### Additional Definitions

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

Aspects of the present disclosure are described herein with reference to flowchart illustrations and/or block diagrams of methods, apparatus (systems), and computer program products according to embodiments of the invention. In some embodiments, the flowchart and block diagrams in the Figures illustrate the architecture, functionality, and operation of possible implementations of systems, methods, and computer program products according to various embodiments of the present invention. In this regard, each block in the flowchart or block diagrams may represent a module, a segment, or a portion of instructions, which may include one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

These computer readable program instructions may be provided to a processor of a general-purpose computer, a special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other devices to cause a series of operational steps to be performed on the computer, other programmable apparatus, or other devices to produce a computer implemented process, such that the instructions which execute on the computer, other programmable apparatus, or other device implement the functions/acts specified in the flowchart and/or block diagram block or blocks.

It will be understood that, although the terms “first,” “second,” etc., may be used herein to describe various elements, components, regions, layers, and/or sections, these elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example embodiments.

Unless specifically stated otherwise, as apparent from the above discussion, it is appreciated that throughout the description, discussions utilizing terms such as “processing,” “performing,” “receiving,” “computing,” “calculating,” “determining,” “identifying,” “displaying,” “providing,” “merging,” “combining,” “running,” “transmitting,” “obtaining,” or the like, refer to the action and processes of a computer system, or similar electronic computing device, that manipulates and transforms data represented as physical (or electronic) quantities within the computer system memories or registers or other such information storage, transmission or display devices.

As used herein, the term “if may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

As used herein, the term “classifiers” refers generally to various types of classifier frameworks, such as neural network classifiers, hierarchical classifiers, ensemble classifiers, etc. In addition, a classifier design can include a multiplicity of classifiers that attempt to partition data into two groups, either organized hierarchically or run in parallel, and then combined to find the best classification. Further, a classifier can include ensemble classifiers wherein a large number of classifiers all attempting to perform the same classification task are learned, but trained with different data/variables/parameters, and then combined to produce a final classification label. The classification methods implemented may be “black boxes” that are unable to explain their prediction to a user (which is the case if classifiers are built using neural networks, for example). The classification methods may be “white boxes” that are in a human-readable form (which is the case if classifiers are built using decision trees, for example). In other embodiments, the classification models may be “gray boxes” that can partially explain how solutions are derived (e.g., a combination of “white box” and “black box” type classifiers).

As used herein, the term “classification” refers to any number or other characters that are associated with a particular property of a sample. The classification can be binary (e.g., positive or negative) or have more levels of classification (e.g., a scale from 1 to 10 or 0 to 1). The term “cutoff” or “threshold” refers to a predetermined number used in an operation. For example, a cutoff value can refer to a classification score as used above. A threshold value may be a value above or below which a particular classification applies. Either of these terms can be used in either of these contexts.

The terms or acronyms like “convolutional neural network,” “CNN,” “neural network,” “NN,” “deep neural network,” “DNN,” “recurrent neural network,” “RNN,” and/or the like may be interchangeably referenced throughout this document.

An “electronic device” or a “computing device” refers to a device that includes a processor and memory. Each device may have its own processor and/or memory, or the processor and/or memory may be shared with other devices as in a virtual machine or container arrangement. The memory will contain or receive programming instructions that, when executed by the processor, cause the electronic device to perform one or more operations according to the programming instructions.

The terms “processor” and “processing device” refer to a hardware component of an electronic device that is configured to execute programming instructions, such as a microprocessor or other logical circuit. A processor and memory may be elements of a microcontroller, custom configurable integrated circuit, programmable system-on-a-chip, or other electronic device that can be programmed to perform various functions. Except where specifically stated otherwise, the singular term “processor” or “processing device” is intended to include both single-processing device embodiments and embodiments in which multiple processing devices together or collectively perform a process.

In this document, the terms “communication link” and “communication path” mean a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices.

The terms “processor” and “processing device” refer to a hardware component of an electronic device that is configured to execute programming instructions, such as a microprocessor or other logical circuit. A processor and memory may be elements of a microcontroller, custom configurable integrated circuit, programmable system-on-a-chip, or other electronic device that can be programmed to perform various functions. Except where specifically stated otherwise, the singular term “processor” or “processing device” is intended to include both single-processing device embodiments and embodiments in which multiple processing devices together or collectively perform a process.

In this document, the terms “communication link” and “communication path” mean a wired or wireless path via which a first device sends communication signals to and/or receives communication signals from one or more other devices. Devices are “communicatively connected” if the devices are able to send and/or receive data via a communication link. “Electronic communication” refers to the transmission of data via one or more signals between two or more electronic devices, whether through a wired or wireless network, and whether directly or indirectly via one or more intermediary devices.

It is noted here that, as used in this specification and the appended claims, the singular forms “a,” “an,” and “the” include plural reference unless the context clearly dictates otherwise.

As used herein, “plurality” means two or more. As used herein, a “set” of items may include one or more of such items.

As used herein, “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like do not necessarily refer to the same embodiment, but may unless the context dictates otherwise.

As used herein, the terms “and/or” or “/” means any one of the items, any combination of the items, or all of the items with which this term is associated.

As used herein, the term “substantially” does not exclude “completely,” e.g., a composition which is “substantially free” from Y may be completely free from Y. Where necessary, the word “substantially” may be omitted from the definition of the present disclosure.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As used herein, the term “each,” when used in reference to a collection of items, is intended to identify an individual item in the collection but does not necessarily refer to every item in the collection. Exceptions can occur if explicit disclosure or context clearly dictates otherwise.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the present disclosure. The upper and lower limits of these smaller ranges may independently be included or excluded in the range, and each range where either, neither, or both limits are included in the smaller ranges is also encompassed within the present disclosure, subject to any specifically excluded limit in the stated range. Where the stated range includes one or both of the limits, ranges excluding either or both of those included limits are also included in the present disclosure.

The use of any and all examples, or exemplary language (e.g., “such as”) provided herein, is intended merely to better illuminate the invention and does not pose a limitation on the scope of the present disclosure unless otherwise claimed. No language in the specification should be construed as indicating any non-claimed element as essential to the practice of the present disclosure.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present disclosure. Nothing herein is to be construed as an admission that the present disclosure is not entitled to antedate such publication by virtue of prior disclosure. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### Examples

A cohort of diabetic patients with VLUs underwent mobile comprehensive vascular assessments, including pre volume plethysmography, ABI/TBI, and PVR. Pre-compression arterial blood flow studies were conducted with patients in a supine or elevated leg position. Standardized care dressings were applied, followed by the application of 3-layer compression wraps. Post-compression arterial volume plethysmography and TBI measurements were taken, and the results were compared with the pre compression ABI/TBI and PVR findings.

Early findings challenged the assumption that ABI/TBI and PVR alone are sufficient indicators for safe compression therapy. The introduction of pre and post volume plethysmography revealed alterations in arterial perfusion in diabetic patients with varying classifications of peripheral arterial disease (PAD). In some cases, compression therapy worsened arterial perfusion, highlighting the need for a more nuanced approach.

This study underscores the importance of precise vascular assessment in diabetic patients with VLUs. While ABI/TBI and PVR remain standard, the inclusion of pre and post volume plethysmography provides a more comprehensive understanding of arterial perfusion dynamics. This nuanced approach allows clinicians to identify patients at risk of worsening arterial perfusion under compression therapy.

### Overview of the Study

### Study Name

Use and Effectiveness of QuantaFlo® PAD Volume Plethysmography in Subjects with Lower Extremity Wound and Ulcers

### Sponsor

### The Wound Pros

### Study Objective

The primary aim of this cross-sectional observational protocol is to investigate the correlation of QuantaFlo® for the detection of PAD (QFPAD) with Ankle Brachial Index (ABI) and Systolic Toe blood pressures (TSP) in a study designed to prove non-inferiority.

### Patient Population

Including Diabetic patients adds a relevant subset to the study population.

By targeting this patient group, the study ensures relevance to real-world scenarios where vascular complications are common.

### Study Design

All subjects will complete the QuantaFlo PAD, ABI, and Toe Systolic Pressure tests. Additionally, Patients with Venous Lower Extremity Ulcers will undergo Pre and Post QuantaFlo Volume Plethysmography and Toe Systolic Pressures (TBP) to determine if there is a change in pressure or QuantaFlo test result.

### Patient Selection

This study will enroll patients who meet the inclusion criteria. The patient involvement in this study is 1 visit lasting about 30 minutes to 1 hour. The overall enrollment period will be 6 months or until 200 patients have been tested. An interim review will occur once 100 patients have been tested.

### Primary Aim

The primary aim of this observational protocol is to investigate the correlation between QuantaFlo® PAD (QFPAD) Volume Plethysmography and traditional methods such as Ankle Brachial Index (ABI) or Systolic Toe blood pressures (TBP) for the detection of Peripheral Arterial Disease (PAD). The study is designed to establish the non-inferiority of QFPAD in identifying flow-limiting PAD to Distal Lower Extremities.

### Primary Hypothesis

The study aims to demonstrate that QFPAD is non-inferior to ABI and/or Systolic Toe Blood Pressures (TBP) in identifying flow-limiting PAD to the Distal Lower Extremities. This hypothesis underscores the core objective of evaluating the effectiveness of QFPAD compared to established diagnostic measure of ABI and TBPs.

### Secondary Hypothesis

The secondary hypothesis focuses on establishing a correlation between QuantaFlo findings and the clinical signs and symptoms of PAD. This secondary aim adds depth to the study by exploring the broader context of how QFPAD aligns with the overall clinical presentation of PAD.

### Exploratory Hypothesis

The exploratory hypothesis delves into the potential benefits of QFPAD in identifying changes in blood flow associated with the use of universal compression therapy in patients with Venous Lower Extremity Ulcers (VLUs). This hypothesis expands the scope of the study by exploring a specific clinical scenario where QFPAD may provide additional insights.

### Inclusion of Diabetic Patients

The inclusion of Diabetic patients requiring Compression Therapy adds a relevant subset to the study population. By targeting this patient group, the study ensures relevance to real-world scenarios where vascular complications are common.

### Inclusion Criteria

The Study will enroll all male and female adult (greater than age 18) patients contracted with The Wound Pros for evaluation and treatment of a lower extremity wound, including those with the Diagnosis of Chronic Lower Extremity VLU in one or both lower Extremities needing Compression Therapy.

### Exclusion Criteria

Any Patient with bilateral lower extremity amputations including or proximal to the trans metatarsal (TMA) level.

### Potential Benefits

a. Improved PAD Diagnosis
b. Clinical Correlation: The secondary hypothesis explores the correlation between QuantaFlo findings and clinical signs/symptoms of PAD. A positive correlation could enhance clinicians' ability to interpret test results in the broader clinical context.
c. Exploratory Insights: The exploratory hypothesis explores the potential benefits of QFPAD in identifying changes in blood flow associated with universal compression therapy in patients with Venous Lower Extremity Ulcers. Positive findings could contribute to understanding utility in specific clinical scenarios.
d. Patient Care Improvement

### Potential Risks

a. Adverse Events: there might be risks associated with the study procedures, such as the administration of the QuantaFlo PAD test, ABI, and TBI.
b. Data Privacy Concerns: Safeguards must be in place to ensure compliance with data privacy protection laws and regulations.
c. Participant Discomfort: Study participants may experience discomfort during certain procedures, such as compression therapy or the various tests administered. Adequate measures should be taken to minimize discomfort.
d. Withdrawal Impact: Participants may choose to withdraw which may impact data integrity.

### Study Method

Patient Screening: During an already established patient visit, the targeted patient population, based on the inclusion and exclusion criteria above, will be presented with the opportunity to participate in the QFPAD study by their provider.

Patients will be presented with a copy of the informed consent form and allowed time to have their questions answered and consider participation in accordance with Good Clinical Practice and the outlined requirements in the Human Subjects section below.

### Study Method Quantaflo

The provider will administer the QuantaFlo PAD Test and record the hemodynamic (blood flow) measurement data from the QFPAD device.

Also, obtain
ABI
TBI
Toe Systolic Pressures (TBP)
   Continue with Treatment Plan

Once all the tests are successfully completed, the Provider will continue with the treatment plan for the wound. If the treatment includes compression wrap of one or both lower extremities, the Provider will obtain a secondary post-compression QFPAD. TBI and Toe Systolic (TBP) measurements on the wrapped extremities. All VLUs in need of compression will utilize Sun Scientific's Aero Wrap gradient compression system with three levels of adjustable compression (20-30 mmHg, 30-40 mmHg, and 40-50 mmHg). The level of compression will be determined by evaluating the ABI. The compression level will be re-adjusted after post-compression QFPAD, and Toe Systolic Pressure have been evaluated.

### Statistical Analysis Plan—SAP

### Pearson's or Spearman Correlation

### Chi-Squared Test

### Study Outcomes

The QuantaFlo PAD test aids clinicians in the diagnosis of vascular disease by measuring blood volume changes using volume plethysmography in the Brachial, Posterior Tibial and Anterior Tibial/Dorsal Pedis arterial distributions and is non-inferior to ABI, TBI or Toe Systolic Pressures

### Secondary Outcome

The QuantaFlo PAD test aids the clinician in the diagnosis of vascular disease by measuring blood volume changes using volume plethysmography in the Brachial, Posterior Tibial and Anterior Tibial/Dorsalis Pedis arterial distribution with an accuracy of 80% or greater.

### Exploratory Outcome

QuantaFlo PAD (QFPAD) aids in the identification of blood flow changes associated when using compression therapy in patients with VLU's.

### Positioning of the Patient

Optimal positioning is supine. If unable to get patient supine, place the patient in a sitting position and elevate legs. Once the patient is comfortable, remove all wound dressings and assess wounds. Take pre-wound debridement pictures

### QuantaFlo PAD

Perform a QuantaFlo on the index fingers of the hand if available. If the index finger is not available or has a wound, proceed to the next best finger. On the lower extremities, try to use the second digit. If not available, choose the next best digit. If the patient has a tremor gently hold the extremity down. Use a lightshield if prompted to by the software to reduce errors. Connect your device to the internet if available to get best results. Make sure to note any pertinent positives or negatives. Also, note the time taken to complete the study.

### Appointment Assessment Vascular

### Vascular Encounter Questionnaire

### Medical History:

1. Do you have a history of diabetes? Y/N
2. Have you been diagnosed with high blood pressure (hypertension)? Y/N
3. Have you ever been told you have high cholesterol? Y/N
4. Have you been diagnosed with heart disease or had any heart-related procedures? Y/N
5. Are you a current or former smoker? Y/N
6. Have you had any previous vascular surgeries or procedures? Y/N
7. Are you taking any blood thinner medications (ie. Aspirin)? If yes, provide details. Y/N
8. Do you have a family history of vascular diseases or circulatory problems? Y/N
9. Do you have a history of amputation(s)? If yes, provide details. Y/N
10. Do you have a history of gangrene? If yes, provide details. Y/N

### Symptoms:

11. Have you experienced pain, cramping, or discomfort in your legs while walking or during physical activity? Describe the location and nature of the pain (intermittent Claudication). Y/N
12. Do you experience leg pain at rest or during the night (rest pain)? Y/N
13. Have you noticed any changes in the color, temperature, or texture of your feet or legs? Y/N
14. Are there any non-healing sores, wounds, or ulcers on your feet or legs? Y/N
15. Have you observed any swelling in your legs, ankles, or feet? Y/N
16. Do you feel numbness, tingling, or a sensation of “pins and needles” in your feet or legs? Y/N

### Physical Examination:

17. Rate your ability to walk without pain on a scale of 0 to 10 (0=no pain, 10=severe pain).
18. Are there any visible varicose veins or spider veins on your legs? Y/N
19. Describe any areas of tenderness or pain in your legs.
20. Are your leg pulses (such as the pulse in your foot or behind the knee) easily palpable? Y/N
21. Have you noticed any hair loss or changes in skin texture on your legs or feet? Y/N

### ABI Testing

ABI checks for narrowed arteries which affects 10% of people over 55.
67% of patients are asymptomatic
Test patients 65+
Test patients 55+ smoking or diabetes
Test all symptomatic patients (intermittent claudication, CLI, non-healing ulcer, wound, neuropathic leg pain

### Contraindications:

Extreme pain in lower extremities
external defibrillator
pacemaker
insulin pump
Bilateral mastectomy
Arterial catheters
Obesity, ankle circumference greater than 13.6 inches
Low perfusion

### 4.4 ABI Compression Guidelines

Perform an Ankle Brachial Index by placing arm and ankle cuffs. Initial cuffs need to cover the dorsalis pedis artery. After the initial run, rotate the cuffs to test the posterior tibial artery.
Normal 1.0-1.3
Vessel Calcifications <1.3: Additional Testing TBI or TSP to determine treatment.
LEAD <0.9-0.8 Proceed with 30-40 mmHg compression, and assess wound healing
0.5<0.8, sustained, high compression (i.e., 30-40 mmHg at the ankle) is not recommended.

### Mixed Venous/Arterial Disease:

ABI is >0.5 to <; 0.8, reduced compression levels (i.e., 23-30 mmHg) are advised
<0.5 compression should be avoided, and the patient referred to a vascular surgeon for surgical evaluation and/or further testing.

### Disinfect Device

ABI training: For vascular technicians, no interpretation of results should be provided to the patient.

### ABI

### Clinical Status

0.95 or > Normal
0.70+/−0.1 Intermittent claudication
0.50+/−0.1 Rest pain
0.30+/−0.1 Impending tissue necrosis
Run the test twice

### Toe Systolic Pressure

Systolic Toe Blood Pressure (TBP) is a measurement of the distal limb systolic blood pressure at the toe. It's particularly useful in patients with noncompressible tibial arteries, such as those with long-standing diabetes, renal failure, or other disorders where vascular calcification is present.

The readings are usually taken at the hallux (big toe). Assessing TBP helps identify or screen for various medical conditions, including:
Blockages in large and small blood vessels
Arterial insufficiency
Cardiac dysfunction
Ischemia (intermittent claudication)
Necrosis and amputation risks

### Standards for TBP Readings:

Normal: 75 mmHg-130 mmHg
Mild occlusion/intermittent claudication/asymptomatic arterial disease: 50 mmHg-74 mmHg
Moderate occlusion/prominent ischemia: 30 mmHg-49 mmHg
Severe occlusion/ischemia/necrosis may be present: <30 mmHg

Select the great toe of each foot if available. If not, choose the next best digit without a wound. Place the PPG sensor distally and the toe cuff proximally on the digit. Access the PPG waveform while raising the cuff pressure with the sphygmometer. Once the PPG waveform flatlines, raise the pressure an additional 20 mmHg. Slowly release the pressure 2-4 mmHg per second until the PPG waveform re-appears. This is the recorded systolic toe pressure.

If compression is not applied, you are done with this part of the research.

### Apply Compression

All VLUs in need of compression will utilize Sun Scientific's Aero Wrap gradient compression system with levels of adjustable compression (20-30 mmHg and 30-40 mmHg).
Original compression determination will be determined by ABI results
ABI 1.4-0.9, TSP 45-55 mmHg, Quantaflo >0.60—Normal 30-40 mmHg
ABI 0.89-0.51. TSP 44-30 mmHg. Quantaflo 0.59-0.30—Mild PAD 20-30 mmHg
ABI 0.5-0.00—Moderate to Severe PAD No compression and referred to a vascular specialist
If ABI<0.50 or TSP<30 mmHg, do not apply compression wrap and refer to Vascular.

Any post-compression readings indicating moderate to severe conditions (QFPAD<0.29, TBI<0.3, TBP<30 mmHg) will be set to 20-30 mmHg and re-evaluated. If severe readings persist, compression will be stopped, and the patient will be referred to a vascular specialist.

If readings improve with compression, the original pressure determination will be maintained. However, if readings worsen, the pressure will be reduced by one category and re-assessed. If poor results persist, compression will be halted, and the patient will be referred to a vascular specialist.

Toe color, foot sensation, and pain will be evaluated during each retesting. Any changes in skin color, sensation loss, or pain will prompt a reduction in compression by one category and re-assessment. If compression is already at the lowest level, it will be discontinued. Patients whose compression is discontinued will be referred to vascular for additional evaluation.

Inflate each compression device to the appropriate setting with 10 pumps before going to the next step.

If the values fall below the following, reduce the compression to the lowest setting and retest. If already at the lowest compression setting and/or the values are still below the displayed values, then remove the compression devices and refer to vascular for additional testing.

### Post Compression QuantaFlo

Perform a post compression Quantaflo following the guidelines outlined in 4.2

### Post Compression Toe Systolic Pressure

Perform a post compression Toe Systolic Pressure following the guideline for TBP readings:
Normal: 75 mmHg-130 mmHg
Mild occlusion/intermittent claudication/asymptomatic arterial disease: 50 mmHg-74 mmHg
Moderate occlusion/prominent ischemia: 30 mmHg-49 mmHg
Severe occlusion/ischemia/necrosis may be present: <30 mmHg
Treat the wound
Treat the wound and determine the applicable VLU care. Do your normal wound care,
compression if needed, determine what compression level to put it at, retest Quantaflo and TSP, if it causes problems with vascular drop to lowest setting, discontinue and refer to vascular, educate patient.
4.10 Refer to Vascular
Refer to vascular if needed and educate the patient if needed.

### Data Entry

Enter your data of the CRF (case report form) and give to Lacey Bauer.

### Documentation

The documentation is the chart will be kept separate from the research documentation.
Screen-shot or record the Quantaflo numbers
Screenshot to remember the numbers of the Quantaflo and to know if the patient needs compression. Export the file to Lacey Bauer.

### Adverse Events—AEs

AE Log.docx

The definition of an Adverse Event (AE) is any untoward medical occurrence associated with the use of an intervention in humans, whether or not it is considered related to the study.

Follow-up with patient 5-7 days after the visit to make sure there are no AEs and SAEs

If there are AEs (any adverse effects from the exams performed) or hospitalizations, let the Research Assistant know to record on AE log or SAE log. The Adverse Event Log includes the severity and relationship to the study procedures (QuantaFlo, Compression Therapy). All reportable AEs will be reviewed by the study PI and reported to the IRB in accordance with IRB Policy and GCP.

### Classify Severity

Mild
Moderate
Severe
Relationship to Study Intervention
Related
Potentially Related
Not Related

### Definitions

### Ankle-Brachial Index (ABI):

The ABI is a widely used test to assess blood flow in the legs. It compares the systolic blood pressure at the ankle to that in the arm.
Interpretation:
Normal: 0.90-1.30
Mild PAD: 0.70-0.89
Moderate PAD: 0.40-0.69
Severe PAD: <0.40
Note that false elevation of ABI values can occur due to arterial wall calcification, especially in diabetic or renal failure patients. In such cases, toe pressures may provide a more accurate assessment1.

### Segmental Pressures:

This test helps localize the location of disease within the leg.
Vertical and horizontal pressure comparisons are used.
Common values:
Aortoiliac disease:
Thigh/Brachial index:
Stenosis: 0.8-1.2
Iliac occlusion: <0.8
SFA (superficial femoral artery) disease:
30 mmHg gradient between high thigh pressure and above-knee pressure indicates disease1.

### Toe Systolic Pressure:

A toe pressure <30 mmHg suggests a probable non-healing lesion.
Digital pressures <80% of brachial pressure indicate proximal disease1.

### QuantaFlo:

QuantaFLo is an accurate PAD testing method that detects even early blood flow volume changes.
QuantaFlo is an accurate PAD testing method that detects even early blood flow volume changes. It outperforms cuff-based ABI, especially in cases with calcified vessels

### Definitions

Arterial insufficiency: Reduced blood flow through arteries can cause tissue damage, leading to symptoms like pain, ulcers and skin changes.

Cyanosis: Bluish discoloration of the skin suggests poor oxygenation that can be related to venous or arterial issues.

Dry skin: Dry skin can exacerbate existing conditions and cause discomfort.

Erythema (redness): Redness often indicates inflammation. It could be related to the rash or other skin conditions.

Lipodermatosclerosis: The condition involves inflammation and hardening of the skin and underlying fat tissue. It's commonly associated with chronic venous insufficiency.

Missing limbs: If you're missing limbs, it's essential to consider how this impacts your skin health and circulation.

Muscle wasting: Muscle atrophy can occur due to various reasons, including reduced blood flow or nerve damage.

Pigmentation: changes below the knee bilaterally: changes in skin color can indicate underlying issues. Chronic venous disease can cause skin discoloration due to poor blood flow.

Rash: Rashes can have various causes, such as allergies, infections, or skin conditions. It's essential to determine the type, location and any associated symptoms.

Spider veins and varicose veins—Both are related to venous issues. Spider veins are small, visible blood vessels near the skin surface while varicose veins are larger, twisted veins.

Trauma: If you've experienced any recent injuries or trauma, it's relevant to consider their impact on your skin and veins.

Unknown mass: An unidentified mass should be evaluated promptly. It's crucial to determine its nature and potential implications.

Venous insufficiency: This condition occurs when veins struggle to return blood to the heart efficiently. It can lead to symptoms like swelling, pain and skin changes.

The present disclosure is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method of providing a compression therapy to a patient who is suffering from venous leg ulcers, comprising:
identifying a venous condition in a patient as having venous leg ulcers and a peripheral arterial disease;
determining an arterial condition of the patient in real-time at least by digital volume plethysmography before and after applying the compression therapy to measure volume changes in the body caused by blood flow using one or more sensors or blood pressure cuffs; and
applying compression pressure to a part of the body of the patient to improve blood circulation based on real-time data of both the venous condition and the arterial condition of the patient, wherein assessment of the arterial condition guides the compression therapy such that the compression therapy does not cause arterial compromise exceeding a pre-determined threshold level.

2. The method of claim 1, wherein the digital volume plethysmography provides real-time vascular assessment in the patient in response to the compression therapy.

3. The method of claim 1, wherein the arterial compromise comprises a blood flow change under compression.

4. The method of claim 1, wherein the digital volume plethysmography is measured in form of digital pressures and/or waveforms.

5. The method of claim 1, comprising modifying an amount of compression pressure based on disease progression of venous leg ulcers.

6. The method of claim 1, wherein the compression pressure is applied through a compression bandage or stocking.

7. The method of claim 1, wherein the compression pressure is applied by a gradient compression system.

8. The method of claim 5, wherein the amount of compression pressure applied to the patient is from 20 mmHg to 30 mmHg or from 30 mmHg to 40 mmHg.

9. The method of claim 1, further comprising determining an amount of compression pressure to the body of the patient by a trained model.

10. The method of claim 9, wherein the trained model comprises a machine learning model.

11. The method of claim 10, wherein the machine learning model comprises a supervised or unsupervised machine learning model.

12. The method of claim 11, wherein the machine learning model comprises Deep Learning algorithm, Logistic Regression, Naive Bayes, Support Vector Machine, Decision Tree, Random Forest, Gradient Boosting, Regularizing Gradient Boosting, K-Nearest Neighbors, a continuous regression approach, Ridge Regression, Kernel Ridge Regression, Support Vector Regression, deep learning approach, Neural Networks, Convolutional Neural Network (CNNs), Recurrent Neural Networks (RNNs), Gated Recurrent Units (GRUs), Long Short Term Memory Networks (LSTMs), Generative Models, Generative Adversarial Networks (GANs), Deep Belief Networks (DBNs), Feedforward Neural Networks, Autoencoders, Variational Autoencoders, Normalizing Flow Models, Deniosing Diffusion Probabilistic Models (DDPMs), Score Based Generative Models (SGMs), Radial Basis Function Networks (RBFNs), Multilayer Perceptrons (MLPs), Stochastic Neural Networks, or a combination thereof.

13. The method of claim 1, wherein the part of the body of the patient comprises a foot of the patient.

14. The method of claim 1, wherein the method is performed in a point-of-care setting.

15. The method of claim 1, wherein the method is performed in a mobile care setting.

16. The method of claim 1, wherein the step of determining the arterial condition comprises determining the arterial condition based on Ankle-Brachial Index (ABI)/Toe-Brachial Index (TBI) and/or by pulse volume recording.

17. The method of claim 1, wherein determining the arterial condition is not performed based on Ankle-Brachial Index (ABI)/Toe-Brachial Index (TBI).

18. The method of claim 1, wherein determining the arterial condition is not performed by pulse volume recording.

19. A system for providing a compression therapy to a patient who is suffering from venous leg ulcers, comprising one or more processors configured to implement the method of claim 1.

20. A non-transitory computer-readable medium comprising instructions that, when executed by one or more processors, cause the one or more processors to perform the method of claim 1.
